(19) United States
(12) Patent Application Publication
(10) Pub. No.: US 2026/0337130 A1
(43) Pub. Date: Oct. 29, 2026

(54) METHOD OF HYDROGENATION

(71) Applicant: KING FAHD UNIVERSITY OF PETROLEUM AND MINERALS, Dhahran (SA)

(72) Inventors: M. Nasiruzzaman SHAIKH, Dhahran (SA); Shahad Musad AL-MUTLAQ, Dhahran (SA); Huda Saeed ALGHAMDI, Dhahran (SA); Ahsan ALI, Dhahran (SA)

(73) Assignee: KING FAHD UNIVERSITY OF PETROLEUM AND MINERALS, Dhahran (SA)

(21) Appl. No.: 19/327,896

(22) Filed: Sep. 12, 2025

Related U.S. Application Data
(60) Provisional application No. 63/793,296, filed on Apr. 23, 2025.

(51) Int. Cl. C07C 5/10

(52) U.S. Cl. CPC C07C 5/10; C07C 2523/656

(57) ABSTRACT

A method of hydrogenation includes contacting toluene with a catalyst and hydrogen gas in a reactor. The catalyst includes a bimetallic cerium(IV) oxide (CeO₂) supported material. A first metal of the bimetallic CeO₂ supported material is rhodium in an amount of 0.01 to 0.05 percent by weight (wt. %). A second metal of the bimetallic CeO₂ supported material is cobalt, iron, manganese, and/or nickel in an amount of 0.05 to 0.5 wt. %. The method includes hydrogenating the toluene to form a hydrogenated product.

### CROSS REFERENCE TO RELATED APPLICATION

This application claims the benefit of U.S. Provisional Application No. 63/793,296, filed Apr. 23, 2025, which is incorporated herein by reference in its entirety.

### STATEMENT OF ACKNOWLEDGEMENT

Support provided by the King Fahd University of Petroleum and Minerals Hydrogen Consortium, King Fahd University of Petroleum and Minerals, Saudi Arabia, through Project H2FC2310 is gratefully acknowledged.

### BACKGROUND

### Technical Field

The present disclosure is directed towards a method of hydrogenation, and more particularly a method of hydrogenation including a catalyst including bimetallic cerium(IV) oxide (CeO₂) supported material.

### Description of Related Art

The “background” description provided herein is for the purpose of generally presenting the context of the disclosure. The work of the presently named inventors, to the extent it is described in this background section, as well as aspects of the description which may not otherwise qualify as prior art at the time of filing, are neither expressly nor impliedly admitted as prior art against the present disclosure.

In an age dominated by the escalating challenges of climate change and the depletion of fossil fuel reserves, the global community faces a decisive turning point. The imperative to shift toward sustainable energy sources has intensified research into alternative fuels. Among alternative fuels, hydrogen stands out as a clean and versatile energy carrier, capable of powering a broad spectrum of applications from transportation to industrial processes. It offers high energy density and emits zero carbon upon use and can be produced through water electrolysis using renewable sources such as solar and wind.

An obstacle to widespread hydrogen adoption is the challenge of storage—specifically, how to store it safely, efficiently, and affordably. Conventional techniques such as high-pressure compression (up to around 700 bar) or cryogenic liquefaction (at approximately −253 °C) raise concerns and require substantial energy input, demand specialized infrastructure, pose safety risks, and struggle to scale cost-effectively.

Liquid organic hydrogen carriers (LOHCs) present a promising alternative. These systems chemically bind hydrogen to an organic liquid, enabling storage under ambient conditions. LOHCs improve safety, leverage existing fuel infrastructure, enable long-distance transport, and eliminate the need for extreme pressures or low temperatures.

Among various LOHC systems, the toluene/methylcyclohexane cycle is attractive an toluene can store about 6.2% hydrogen by weight, demonstrates excellent thermal stability, has low volatility, and is compatible with current logistics, making the system scalable and economically viable.

Hydrogenation of toluene to methylcyclohexane typically relies on noble-metal catalysts such as platinum, palladium, ruthenium, or rhodium supported on oxides such as alumina or ceria. While high performance has been achieved under moderate temperatures and pressures, the dependence on expensive precious metals and challenges in catalyst stability and scale continue to limit practical deployment.

Accordingly, an object of the present disclosure is directed to a metal-free nickel-based catalytic composition, which exhibits enhanced activity and selectivity for aromatic hydrogenation reactions under mild conditions while avoiding the use of expensive platinum-group metals, thereby overcoming the limitations of the prior art.

### SUMMARY

In an exemplary embodiment, a method of hydrogenation is described. The method includes contacting toluene with a catalyst and hydrogen gas in a reactor. The catalyst includes a bimetallic cerium(IV) oxide (CeO₂) supported material. A first metal of the bimetallic CeO₂ supported material is rhodium in an amount of 0.01 to 0.05 percent by weight (wt. %). A second metal of the bimetallic CeO₂ supported material is selected from the group consisting of cobalt, iron, manganese, and nickel in an amount of 0.05 to 0.5 wt. %. The method includes hydrogenating the toluene to form a hydrogenated product.

In some embodiments, the contacting occurs at a temperature of 60 to 140 °C.

In some embodiments, the contacting occurs at a pressure of 20 to 60 bar.

In some embodiments, a weight ratio of the toluene to the catalyst is 15:1 to 20:1.

In some embodiments, 60 to 85% of the toluene is hydrogenated.

In some embodiments, the reactor includes a stirrer.

In some embodiments, the stirrer has a rotational speed of up to 2500 rpm.

In some embodiments, at least 98% of the hydrogenated product is methylcyclohexane.

In some embodiments, the method further includes heating the hydrogenated product at a temperature of 180 to 270 °C to dehydrogenate the hydrogenated product.

In some embodiments, the heating occurs for at least 8 hours.

In some embodiments, the catalyst is in the shape of agglomerated particles having a particle circumference of 230 to 260 μm.

In some embodiments, the rhodium, the cobalt, the iron, the manganese, and the nickel have an oxidation state of zero.

In some embodiments, the second metal of the bimetallic CeO₂ supported material is manganese, and 80 to 90% of the toluene is hydrogenated.

In some embodiments, the second metal of the bimetallic CeO₂ supported material is cobalt, and 75 to 80% of the toluene is hydrogenated.

In some embodiments, the second metal of the bimetallic CeO₂ supported material is nickel, and 65 to 70% of the toluene is hydrogenated.

In some embodiments, the second metal of the bimetallic CeO₂ supported material is iron, and 62 to 66% of the toluene is hydrogenated.

In some embodiments, the method further includes repeating the contacting to form the hydrogenated product and repeating the heating to dehydrogenate the hydrogenated product. The repeating occurs at least three times. Forming the hydrogenated product has a conversion rate of at least 80% and dehydrogenating the hydrogenated product has a conversion rate of at least 80%. Conversion rate is based on weight of the toluene and the hydrogenated product.

In some embodiments, the agglomerated particles include nanoparticles having a longest dimension of 10 to 100 nm.

In some embodiments, the heating occurs for at least 24 hours and at least 80% of the hydrogenated product is dehydrogenated.

In another embodiment, a process of making the catalyst is described. The process includes dissolving a rhodium salt and a salt selected from the group consisting of a cobalt salt, an iron salt, a manganese salt, and a nickel salt in water to form a first solution. The process includes adding cerium oxide to the first solution to form a second solution and heating the second solution to a temperature of 50 to 70 °C for 60 to 120 minutes. The process includes adding a base to the second solution to form the catalyst.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a schematic flowchart of a method of hydrogenation, according to certain embodiments.

FIG. 1B is a schematic flowchart of a method of making a catalyst, according to certain embodiments.

FIG. 1C is a schematic diagram of an experimental setup, according to certain embodiments.

FIG. 2 is an X-ray diffraction (XRD) pattern for fresh catalysts supported on CeO₂, according to certain embodiments.

FIG. 3A is a scanning electron microscopy (SEM) analysis for a rhodium (Rh)/cerium(IV) oxide (CeO₂) supported catalyst, according to certain embodiments.

FIG. 3B is an SEM analysis for a Rh and manganese (Mn)/CeO₂ supported catalyst, according to certain embodiments.

FIG. 3C is an SEM analysis for a Rh and cobalt (Co)/CeO₂ supported catalyst, according to certain embodiments.

FIG. 4A is a transmission electron microscopy (TEM) analysis for Rh/CeO₂ supported catalyst, according to certain embodiments.

FIG. 4B is a TEM analysis for RhMn/CeO₂ supported catalyst, according to certain embodiments.

FIG. 4C is a TEM analysis for RhCo/CeO₂ supported catalyst, according to certain embodiments.

FIG. 5 is a bar graph showing catalytic activity of various catalysts for hydrogenation of toluene, according to certain embodiments.

FIG. 6 depicts the effect of reaction temperature on hydrogenation of toluene, according to certain embodiments.

FIG. 7 depicts the effect of reaction pressure on hydrogenation of toluene, according to certain embodiments.

FIG. 8 depicts the effect of catalyst dosage (weight (mg)) on hydrogenation of toluene, according to certain embodiments.

FIG. 9A depicts dehydrogenation of methylcyclohexane over time, according to certain embodiments.

FIG. 9B depicts dehydrogenation of methylcyclohexane at various temperatures, according to certain embodiments.

FIG. 10A depicts stability testing of RhMn/CeO₂ for hydrogenation of toluene, according to certain embodiments.

FIG. 10B depicts stability testing of the RhMn/CeO₂ for dehydrogenation of methylcyclohexane (MCH), according to certain embodiments.

### DETAILED DESCRIPTION

When describing the present disclosure, the terms used are to be construed in accordance with the following definitions, unless a context dictates otherwise.

Embodiments of the present disclosure will now be described more fully hereinafter with reference to the accompanying drawings wherever applicable, in that some, but not all, embodiments of the disclosure are shown.

In the drawings, like numbered reference numerals will be used to designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term ‘room temperature’ refers to a temperature range of 25±3 degrees Celsius (°C).

As used herein, the term “hydrogenation” refers to a chemical reaction in which hydrogen (H₂) is added to a compound, typically in the presence of a catalyst, resulting in the saturation of double or triple bonds within organic molecules such as alkenes or alkynes.

As used herein, the term “catalyst” refers to a substance that increases the rate of a chemical reaction without itself undergoing permanent chemical change or consumption in the reaction process. A catalyst make work to lower the activation energy of a reaction thereby increasing the rate of the reaction.

As used herein, the term “oxidation state” refers to the numerical value assigned to an atom within a molecule or ion that represents the number of electrons lost or gained by that atom, as determined by a set of standardized rules, for the purpose of tracking electron transfer in chemical reactions.

Aspects of the present disclosure are directed to the efficiency of a catalyst including a bimetallic CeO₂ supported material in reversible hydrogenation and dehydrogenation reactions, underscoring efficient and selective processes in sustainable energy storage applications.

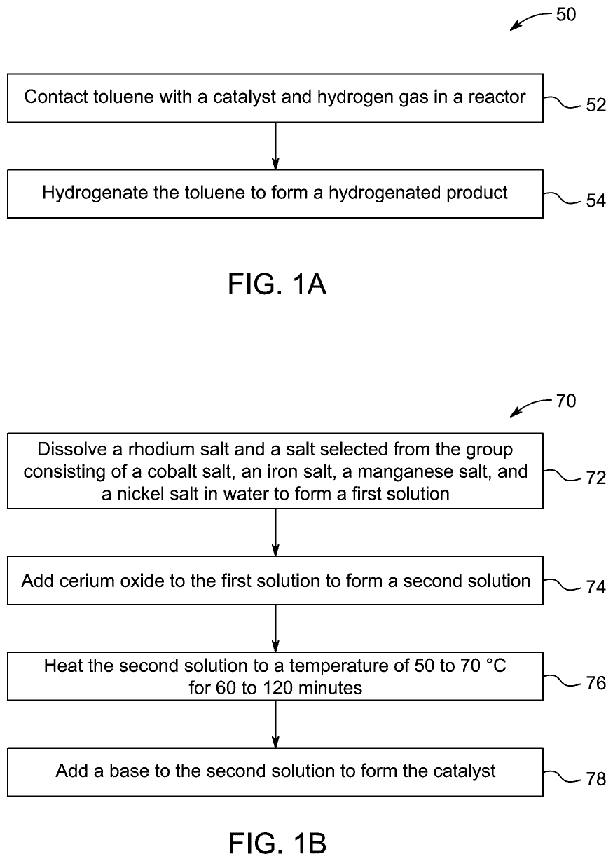

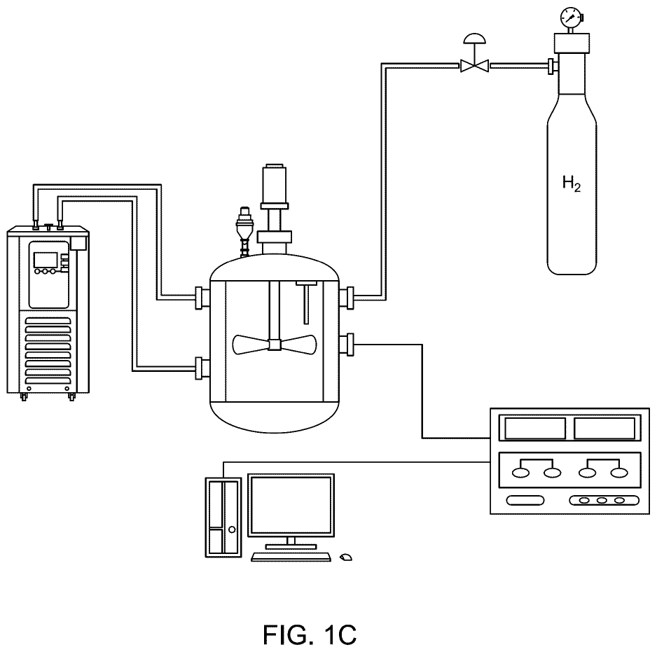

FIG. 1A illustrates a flow chart of a method 50 of hydrogenation. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes contacting toluene with a catalyst and hydrogen gas in a reactor. In some embodiments, a weight ratio of the toluene to the catalyst is 15:1 to 20:1, preferably 16:1 to 19:1, more preferably 17:1 to 18:1, and yet more preferably about 17.3:1. In some embodiments, the reactor may include tube furnace reactors, fluidized bed reactors, screw-conveyor reactors, liquefaction autoclave reactors, catalytic bubbling fluidized-bed reactors, integrated stirred type chemical reactors, a combination thereof, and any reactors known in the art. In some embodiments, the reactor includes a stirrer. In some embodiments, the stirrer may include, but is not limited to, a bladed propeller (e.g., a two-bladed propeller, a three-bladed propeller, a four-bladed propeller, etc.), a simple paddle, an anchor blade, a frame blade, a wiper blade, an open blade, a vane-type blade, an umbrella-type blade, a helical ribbon, an axial turbine, a radial turbine, a screw with a draft tube, a combination thereof, and the like. In a preferred embodiment, the reactor includes a stirrer, and the stirrer is a blade-type stirrer. In some embodiments, the reactor includes a stirrer, and the stirrer is powered by a magnet. In other embodiments, the reactor includes a stirrer, and the stirrer is powered by any source known in the art. In some embodiments, the stirrer has a rotational speed of up to 2500 rpm, preferably up to 2250 rpm, preferably up to 2000 rpm, preferably up to 1500 rpm, and preferably up to 1000 rpm. In alternative embodiments, the stirrer may have a rotational speed of up to 2500 rpm, preferably up to 3000 rpm, preferably up to 3500 rpm, preferably up to 4000 rpm, preferably up to 4500 rpm, and preferably up to 5000 rpm. In other embodiments, the stirrer may have a varying rotational speed. In some embodiments, the contacting occurs at a temperature of 60 to 140 °C, preferably 65 °C to 135 °C, preferably 70 °C to 130 °C, preferably 75 °C to 125 °C, preferably 80 to 120 °C, preferably 85 to 115 °C, preferably 90 to 110 °C, and preferably 95 to 105 °C. In some embodiments, the contacting occurs at a pressure of 20 to 60 bar, preferably 25 bar to 55 bar, preferably 30 bar to 50 bar, preferably 35 bar to 45 bar, and preferably about 40 bar.

The catalyst includes a bimetallic cerium(IV) oxide (CeO₂) supported material. A first metal of the bimetallic CeO₂ supported material is rhodium in an amount of 0.01 to 0.05 percent by weight (wt. %), preferably 0.015 to 0.045 wt. %, preferably 0.02 to 0.04 wt. %, and preferably 0.025 to 0.035 wt. %. A second metal of the bimetallic CeO₂ supported material is selected from the group consisting of cobalt, iron, manganese, and nickel in an amount of 0.05 to 0.5 wt. %, preferably 0.1 to 0.45 wt. %, preferably 0.15 to 0.4 wt. %, preferably 0.2 to 0.35 wt. %, and preferably 0.25 to 0.3 wt. %.

In some embodiments, the catalyst is in the shape of agglomerated particles having a particle circumference of 230 to 260 μm, preferably 235 μm to 255 μm, preferably 240 μm to 250 μm, preferably 241 μm to 249 μm, preferably 242 μm to 248 μm, preferably 243 μm to 247 μm, more preferably 244 μm to 246 μm, and yet more preferably about 244.291 μm. In some embodiments, the agglomerated particles include nanoparticles having the longest dimension of 10 to 100 nm, preferably 15 to 95 nm, preferably 20 to 90 nm, preferably 25 to 85 nm, preferably 30 to 80 nm, preferably 35 to 75 nm, preferably 40 to 70 nm, preferably 45 to 65 nm, and preferably 50 to 60 nm. In some embodiments, the nanoparticles are on a surface of the agglomerated particles. In other embodiments, the nanoparticles are connected to one another to form the agglomerated particles. In some embodiments, the rhodium, the cobalt, the iron, the manganese, and the nickel have an oxidation state of zero. In some embodiments, at least 50%, preferably at least 60%, preferably at least 70%, preferably at least 80%, preferably at least 90%, preferably at least 95%, and preferably at least 99% of the rhodium, the cobalt, the iron, the manganese, and the nickel have an oxidation state of zero based on a total amount of the rhodium, the cobalt, the iron, the manganese, and the nickel. In alternative embodiments, less than 5%, preferably less then 4%, preferably less than 3%, preferably less than 2%, preferably less than 1%, preferably less than 0.5%, and preferably less than 0.1% of the rhodium, the cobalt, the iron, the manganese, and the nickel have an oxidation state that is not zero (i.e., +1, +2, +3, +4, and the like) based on a total amount of the rhodium, the cobalt, the iron, the manganese, and the nickel.

At step 54, the method 50 includes hydrogenating the toluene to form a hydrogenated product. In some embodiments, 60 to 85%, preferably 65 to 80%, and preferably 70 to 75% of the toluene is hydrogenated. In one embodiment, about 84% of the toluene is hydrogenated. In another embodiment, about 81% of the toluene is hydrogenated. In another embodiment, about 77% of the toluene is hydrogenated. In one embodiment, about 68% of the toluene is hydrogenated. In yet another embodiment, about 64% of the toluene is hydrogenated. In some embodiments, the toluene is hydrogenated to methylcyclohexane. In some embodiments, at least 98%, preferably 99%, preferably 99.5%, and preferably 99.9% of the hydrogenated product is methylcyclohexane. In some embodiments, the toluene is hydrogenated partially to 1-methylcyclohexene, 1-methylcyclohexa-1,3-diene, and the like.

In some embodiments, the method further includes heating the hydrogenated product at a temperature of 180 to 270 °C, preferably 185 to 265 °C, preferably 190 to 260 °C, preferably 195 to 255 °C, preferably 200 to 250 °C, preferably 205 to 245 °C, preferably 210 to 230 °C, and preferably 215 to 225 °C to dehydrogenate the hydrogenated product. In one embodiment, the hydrogenated product is methylcyclohexane and the method further includes dehydrogenating the methylcyclohexane to for a dehydrogenated product (for example, toluene). In some embodiments, the heating occurs for at least 8 hours, preferably at least 10 hours, preferably at least 12 hours, preferably at least 14 hours, preferably at least 16 hours, preferably at least 18 hours, preferably at least 20 hours, preferably at least 22 hours, and preferably at least 24 hours. In some embodiments, the heating occurs for at least 24 hours and at least 80%, preferably at least 85%, preferably at least 90%, preferably at least 95%, and preferably at least 99% of the hydrogenated product is dehydrogenated.

In some embodiments, the second metal of the bimetallic CeO₂ supported material is manganese, and 80 to 90%, preferably 81 to 89%, preferably 82 to 88%, preferably 83 to 87%, more preferably 83.5 to 86%, and yet more preferably about 84% of the toluene is hydrogenated. In some embodiments, the second metal of the bimetallic CeO₂ supported material is cobalt, and 75 to 80%, preferably 76 to 79%, more preferably 76.5 to 78%, and yet more preferably about 77% of the toluene is hydrogenated. In some embodiments, the second metal of the bimetallic CeO₂ supported material is nickel, and 65 to 70%, preferably 66 to 69%, more preferably 67 to 68.5%, and yet more preferably about 68% of the toluene is hydrogenated. In some embodiments, the second metal of the bimetallic CeO₂ supported material is iron, and 62 to 66%, preferably 63 to 65%, more preferably 63.5 to 64.5%, and yet more preferably about 64% of the toluene is hydrogenated.

In some embodiments, the method further includes repeating the contacting to form the hydrogenated product and repeating the heating to dehydrogenate the hydrogenated product. In some embodiments the repeating occurs at least three times, preferably at least four times, and preferably five times. In some embodiments, forming the hydrogenated product has a conversion rate of at least 80%, preferably at least 85%, preferably at least 90%, preferably at least 95%, and preferably at least 99%, dehydrogenating the hydrogenated product has a conversion rate of at least 80%, preferably at least 85%, preferably at least 90%, preferably at least 95%, and preferably at least 99%. In a preferred embodiment, conversion rate is based on weight of the toluene and the hydrogenated product. In some embodiments, the repeating can occur for any number of times.

FIG. 1B illustrates a schematic flow chart of a process 70 of making the catalyst. The order in which the process 70 is described is not intended to be construed as a limitation, and any number of the described process steps can be combined in any order to implement the process 70. Additionally, individual steps may be removed or skipped from the process 70 without departing from the spirit and scope of the present disclosure.

At step 72, the process 70 includes dissolving a rhodium salt and a salt selected from the group consisting of a cobalt salt, an iron salt, a manganese salt, and a nickel salt in water to form a first solution. In some embodiments, the rhodium salt may include, but is not limited to, rhodium(III) chloride, rhodium(II) chloride, rhodium(III) nitrate, rhodium(II) acetate (dimer), rhodium(III) oxide, rhodium-platinum oxide (nishimura's catalyst), chlorotris(triphenylphosphine)rhodium(I) (wilkinson's catalyst), rhodium chloride-cyclooctadiene dimer, rhodium acetylacetonate-cyclooctadiene complex, a combination thereof, and the like. In a preferred embodiment, the rhodium salt is rhodium(II) chloride. In some embodiments, the cobalt salt may include, but is not limited to, cobalt(II) oxide, cobalt(III) oxide, cobalt(II) chloride, cobalt(II) fluoride, cobalt(II) bromide, cobalt(II) iodide, cobalt(II) sulfide, cobalt(II) nitrate, cobalt(II) acetate, a combination thereof, and the like. In some embodiments, the iron salt may include, but is not limited to, iron(II) oxide, iron(III) oxide, iron(II) chloride, iron(II) fluoride, iron(II) bromide, iron(II) iodide, iron(II) sulfide, iron(II) nitrate, iron(II) acetate, a combination thereof, and the like. In some embodiments, the manganese salt may include, but is not limited to, manganese(II) oxide, manganese(III) oxide, manganese(II) chloride, manganese(II) fluoride, manganese(II) bromide, manganese(II) iodide, manganese(II) sulfide, manganese(II) nitrate, manganese(II) acetate, a combination thereof, and the like. In some embodiments, the nickel salt may include, but is not limited to, nickel(II) oxide, nickel(III) oxide, nickel(II) chloride, nickel(II) fluoride, nickel(II) bromide, nickel(II) iodide, nickel(II) sulfide, nickel(II) nitrate, nickel(II) acetate, a combination thereof, and the like. In some embodiments, the water may be tap water, distilled water, bi-distilled water, deionized water, deionized distilled water, reverse osmosis water, hard water, fresh water, brine/salt water, a combination thereof, and the like. In some embodiments, the hard water and the freshwater may include salts of sodium, magnesium, calcium, potassium, ammonium, and iron, anions such as chloride, bicarbonate, carbonate, sulfate, sulfite, phosphate, iodide, nitrate, acetate, citrate, fluoride, and nitrite, a combination thereof, and the like. In a preferred embodiment, the water is deionized water.

At step 74, the process 70 includes adding cerium oxide to the first solution to form a second solution. In some embodiments, the cerium oxide may be added to the first solution by methods such as precipitation (co-precipitation), hydrothermal, sol-gel, solvothermal, spray pyrolysis, ball milling (mechanical), microwave-assisted synthesis, micro-emulsification, pechini process, ultrasound-assisted precipitation, a combination thereof, and the like. In a preferred embodiment, the method of addition is ultrasonication. As used herein, the term “ultrasonication” refers to a process in which sound energy is applied to a sample to agitate particles within the sample.

At step 76, the process 70 includes heating the second solution to a temperature of 50 to 70 °C, preferably 53 to 67 °C, preferably 55 to 65 °C, more preferably 57 °C to 63 °C, and yet more preferably about 60 °C for 60 to 120 minutes (min), preferably 65 to 115 min, preferably 70 to 110 min, preferably 75 to 105 min, preferably 80 to 100 min, more preferably 85 to 95 min, and yet more preferably about 90 min. In some embodiments, the heating can be performed by using heating appliances such as ovens, microwaves, autoclaves, hot plates, heating mantles and tapes, oil baths, salt baths, sand baths, air baths, hot-tube furnaces, hot-air guns, a combination thereof, and the like.

At step 78, the process 70 includes adding a base to the second solution to form the catalyst. The base may include, but is not limited to, an alkaline earth metal hydroxide such as beryllium hydroxide (Be(OH)₂), magnesium hydroxide (Mg(OH)₂), strontium hydroxide (Sr(OH)₂), and calcium hydroxide (Ca(OH)₂), and an alkali metal hydroxide such as lithium hydroxide (LiOH), sodium hydroxide (NaOH), potassium hydroxide (KOH) and rubidium hydroxide (RbOH), and cesium hydroxide (CsOH). In a preferred embodiment, the base is sodium borohydride (NaBH₄).

### EXAMPLES

The following examples describe and demonstrate a method of hydrogenation. The examples are provided solely for illustration and are not to be construed as limitations of the present disclosure, as many variations thereof are possible without departing from the spirit and scope of the present disclosure.

### Example 1: Materials

All chemicals were purchased from Sigma Aldrich and used as received. For Fourier-transform infrared (FTIR) spectroscopy, KBr was used as the IR transparent window material on a Nicolet 720, which has a wavenumber range of 500 to 4000 cm⁻¹. X-ray diffraction (XRD) data were acquired using a Rigaku Ultima-IV diffractometer and Cu—K radiation (1.5405) at 40 kV and 25 mA over a 2θ range from 5° to 90°. Samples for scanning electron microscopy (SEM) were fabricated from ethanolic solutions on alumina stubs and gold-coated using an automatic gold coater (Quorum, Q150T E). For elemental analysis and mapping, energy-dispersive X-ray spectra (EDS) were produced using a Lyra 3 attachment to the SEM.

Initially, in a round bottom flask, 0.5 wt. % of Rh was utilized by dissolving 1 mg of RhCl₂, and 10 wt. % of transition metal (Co, Fe, Mn, and Ni) in 20 mL of deionized water using ultra sonication, to ensure the metal dissociation process. 100 mg of the support (CeO₂) was added to the mixture and stirred by a magnetic stirrer and heated at 60 °C for 90 min. Then, 30 mg of NaBH₄, the reducing agent, was added to the mixture to reduce the metal to zero state. The material was then collected via centrifuge and washed three times, then vacuumed for 24 hours. Finally, it was kept in an inert environment.

The hydrogenation and dehydrogenation experiments were carried out in an integrated stirred type chemical reactor with a volume of 100 mL, as shown in FIG. 1C. The reactor was fabricated using stainless steel and its maximum operating temperature and pressure were 300 °C and 15 MPa. The heater was fixed around the reactor to provide uniform heating with a maximum operating temperature of 760 °C and an O-ring was used for proper sealing. The reactor houses a blade-type stirrer capable of reaching speeds up to 2000 rpm. The stirrer was magnetically powered by a direct current (DC) motor from Bodine electric company, S/N 6037QQIA0016, with a maximum rotation speed of 2500 rpm. The stirrer was cooled during the experiment using a water chiller so that it does not lose its magnetic properties at higher operating temperatures. The reactor and heater temperature were monitored by installing two K-type thermocouples of 1.58 mm thickness. The pressure transducer was installed to examine the pressure during the reaction.

2 mL of toluene and 100 mg of catalyst were introduced into the reactor. Subsequently, the reactor was sealed and purged with hydrogen gas three times to remove any residual air inside, ensuring an inert atmosphere for the experiment. The required temperature and stirring rate for the experiment were controlled using the system controller. Hydrogen gas was then introduced into the reactor until the requisite pressure and heater was turned on.

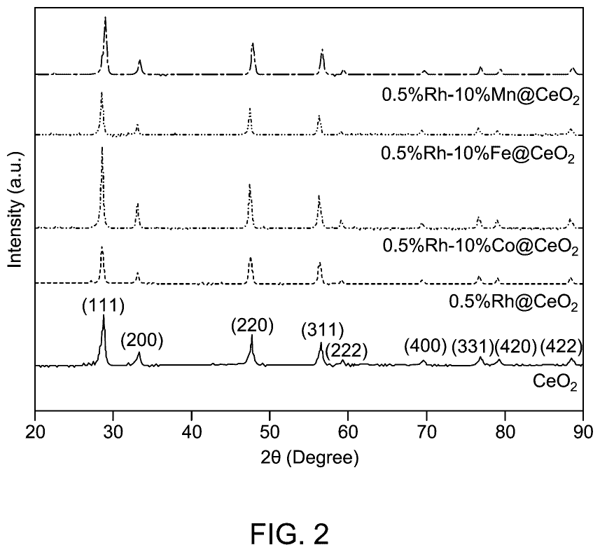

XRD analysis of CeO₂ and its modified forms revealed structural changes upon the addition of various dopants, as shown in FIG. 2. Pure CeO₂ exhibited a prominent peak around 28 degrees, corresponding to the (111) plane. When 0.5 wt. % Rh was introduced, the peak at 28° was enhanced, indicating increased crystallinity or phase concentration due to Rh incorporation or lattice distortion. Further addition of 10 wt. % Co along with Rh resulted in even greater enhancement of the 28-degree peak and a new enhancement at 48 degrees associated with the (220) plane. This indicates a synergistic effect of Rh and Co in stabilizing the CeO₂ structure and possibly forming new phases. In contrast, the addition of Fe and Mn did not cause large shifts or enhancements in the XRD peaks, indicating that these elements do not greatly alter the CeO₂ crystal structure. The stability of the CeO₂ lattice with Fe and Mn additions indicates minimal integration into the lattice or formation of amorphous or dispersed phases. These findings indicate interactions between CeO₂ and different dopants, with Rh and Co enhancing crystallinity and structural reorganization, while Fe and Mn maintain the original lattice structure.

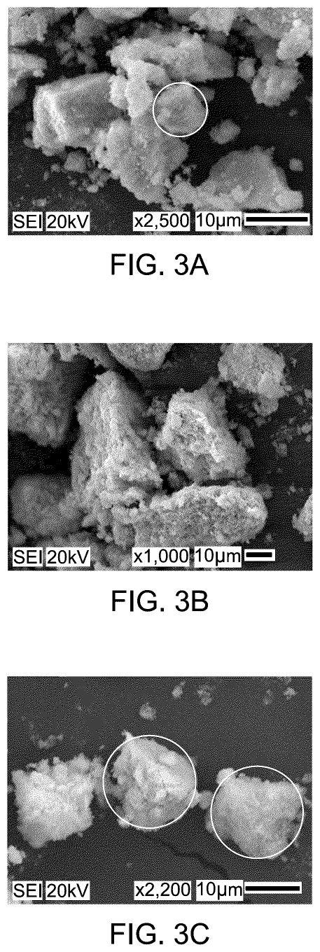

The SEM analysis provided insights into the morphological changes of the catalysts, particularly focusing on agglomeration and particle size variations upon the addition of Mn and Co to the 0.5% Rh/CeO₂ system, as depicted in FIGS. 3A-3C. Initially, the 0.5% Rh/CeO₂ catalyst exhibited a relatively uniform particle size of approximately 65.234 μm; however, when Mn and Co were introduced into the 0.5% Rh/CeO₂ catalyst, agglomeration was observed. An increase in particle size to 244.291 μm was observed for 0.5% Rh10% Co/CeO₂. This increase in particle size can be attributed to the agglomerative effect induced by the presence of Co. Addition of the Co, Fe, Mn, and Ni facilitated the clustering of particles, reducing the overall surface energy and leading to larger agglomerates. Co and Mn can act as binding agents, promoting the coalescence of smaller particles into larger aggregates.

Inductively coupled plasma (ICP) analysis results for the catalysts reveal elemental loadings, as presented in Table 1. For the 0.5 wt. % Rh/CeO₂ catalyst, the ICP results confirm the presence of 4.44 wt. % Ce and 0.0325 wt. % Rh, indicating an incorporation of Rh. When 10 wt. % Co is added to the 0.5 wt. % Rh/CeO₂ catalyst, the composition is 4.69 wt. % Ce, 0.031 wt. % Rh, and 0.14 wt. % Co. For the 0.5 wt. % Rh 10 wt. % Mn/CeO₂ catalyst, the ICP analysis displays 3.85 wt. % Ce, 0.030 wt. % Rh, and 0.243 wt. % Mn, indicating addition of Mn and Rh. Addition of Ni and Rh results in a 0.5 wt. % Rh 10 wt. % Ni/CeO₂ catalyst, with 3.85 wt. % Ce, 0.031 wt. % Rh, and 0.173 wt. % Ni, demonstrating successful incorporation of Ni and Rh.

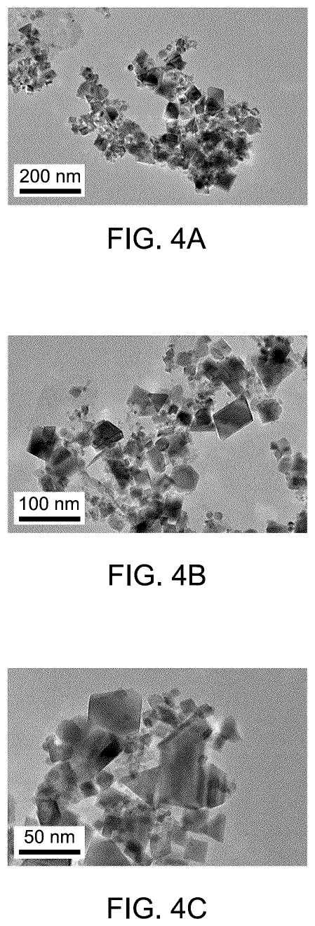

The TEM images, shown in FIGS. 4A-4C, illustrate the morphology and size distribution of the catalysts at varying magnifications. For 0.5% Rh/CeO₂, agglomerated clusters are developed with a mix of irregular shapes, as seen in FIG. 4A. FIG. 4B reveals that the bimetallic catalyst (0.5% Rh10% Mn/CeO₂) at a higher magnification with a 100 nm scale bar includes defined, predominantly cubic or near-cubic particles, though some aggregation persists. For 0.5% Rh10% Co/CeO₂, the particles' crystalline nature with clear, well-defined edges is detailed in FIG. 4C. The morphological analysis indicates a successful synthesis of nanoparticles with uniform shapes and sizes and some degree of agglomeration.

TABLE 1
ICP analysis results for the various CeO₂ supported catalysts

| Sample ID | Ce | Rh | Co | Fe | Mn | Ni |
|---|---|---|---|---|---|---|
| Unit | mg/kg | mg/kg | mg/kg | mg/kg | mg/kg | mg/kg |
| PQL | 1.3 | 0.06 | 0.1 | 1.3 | 0.05 | 0.6 |
| 0.5% Rh@CeO₂ | 44400.0 | 325.6 | <2.1 | <1.3 | 0.0 | 0.0 |
| 0.5% Rh-10% Co@CeO₂ | 46990.0 | 310.1 | 1402.0 | 0.0 | 0.0 | <12.1 |
| 0.5% Rh-10% Fe@CeO₂ | 44900.0 | 267.9 | 0.0 | 1650.0 | <4.2 | 0.0 |
| 0.5% Rh-10% Mn@CeO₂ | 38530.4 | 305.3 | 0.0 | 0.0 | 2432.4 | 0.0 |
| 0.5% Rh-10% Ni@CeO₂ | 38530.4 | 315.1 | 0.0 | 0.0 | 0.0 | 173 |

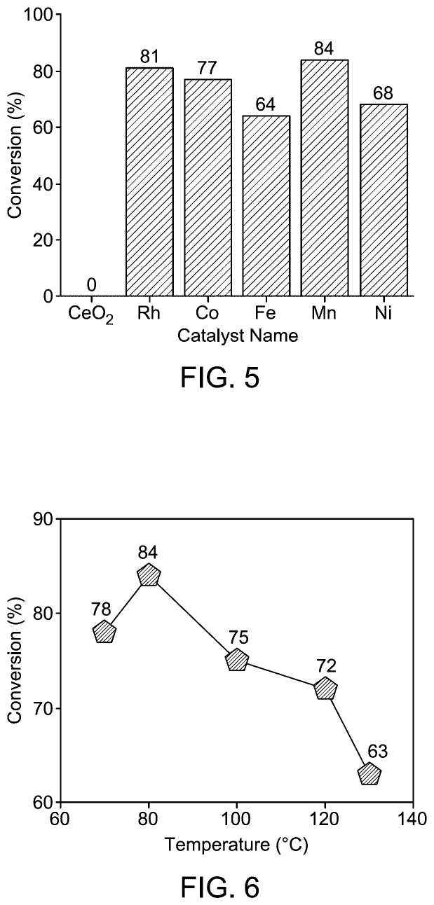

The catalytic activity for the hydrogenation of toluene was investigated employing various catalyst compositions as depicted in FIG. 5. Initially, experiments were conducted at 80 °C and 40 bars, employing different catalyst combinations. Among the tested catalysts, 0.5Rh-10% Mn/CeO₂ emerged as the most effective, exhibiting a toluene conversion of 84% was achieved with a selectivity of >99% towards methylcyclohexane (MCH). The 0.5Rh-10% Fe/CeO₂ catalyst demonstrated comparatively lower catalytic activity, yielding a toluene conversion of 64% under the same conditions. The monometallic catalyst 0.5% Rh/CeO₂ yielded a toluene conversion of 81%, which was lower in comparison to 0.5Rh-10% Mn/CeO₂. 0.5% Rh/CeO₂ exhibited higher toluene conversion rate when compared to 0.5Rh-10% Co/CeO₂, 0.5Rh-10% Ni/CeO₂, and 0.5Rh-10% Fe/CeO₂, yielding toluene conversions of 77%, 68%, and 64%, respectively. The catalytic activity order of the catalysts was as follows: 0.5Rh-10% Mn/CeO₂>0.5% Rh/CeO₂>0.5Rh-10% Co/CeO₂>0.5Rh-10% Ni/CeO₂>0.5Rh-10% Fe/CeO₂. These findings underscore the enhancement of catalytic activity imparted by Mn and the diminished performance associated with Fe incorporation in the catalyst composition.

The catalytic activity of 0.5Rh-10% Mn/CeO₂ was assessed over a temperature range from 70 to 130 °C, as shown in the FIG. 6. The results elucidate that the toluene conversion increases from 78% at 70 °C to a maximum of 84% at 80 °C, highlighting a desired temperature for this catalyst; however, as the temperature is further increased, a decline in conversion efficiency is observed, with conversion rates dropping to 75% at 100 °C, 72% at 120 °C, and reaching a low of 63% at 130 °C. This decrease in activity beyond 80 °C indicates a deterioration in catalyst performance at higher temperatures. Several factors may contribute to the observed decline in catalytic activity. Catalyst deactivation may occur due to sintering, which can reduce the active surface area of the catalyst, or thermal degradation, which can lead to the loss of active sites.

Another contributing factor could be the thermal desorption of reactants from the catalyst surface, preventing them from participating in the reaction and resulting in lower conversion rates. These findings underscore the importance of temperature control in catalytic processes to maintain high efficiency. The desired temperature for this catalyst is around 80 °C, where the conversion rate is the highest.

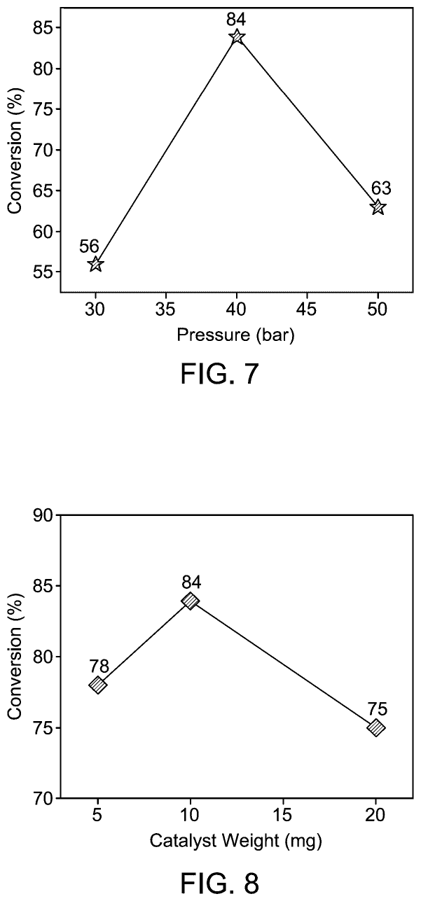

Experiments were undertaken to investigate the influence of reaction pressure on the hydrogenation of toluene. The reaction pressure was systematically varied within the range of 30 to 50 bars at while maintaining a constant temperature of 80 °C. There is a correlation between pressure and toluene conversion, as shown in FIG. 7. Specifically, as the pressure was increased from 30 to 40 bars, toluene conversion exhibited an improvement, increasing from 56% to 84%, respectively; however, further elevation of the pressure to 50 bars resulted in a decline in catalytic activity, with toluene conversion diminishing to 63%. This observed reduction in performance at higher pressures indicates a limitation attributed to catalyst instability under elevated pressure conditions.

The impact of catalyst dosage on the hydrogenation of toluene was investigated by systematically varying the amount of catalyst within the range of 5 mg to 20 mg while maintaining a constant toluene amount of 1 mL. Experiments were conducted under standard conditions of 80 °C and 40 bars pressure. Analysis of the results revealed a trend where toluene conversion exhibited a positive correlation with increasing catalyst dosage, as shown in FIG. 8. Specifically, as the dosage was augmented from 5 mg to 10 mg, toluene conversion demonstrated an enhancement, rising from 78% to 84%; however, a subsequent increase in dosage to 20 mg led to a decline in toluene conversion to 75%. These observations indicate interplay between catalyst dosage and catalytic activity, highlighting the necessity for dosage regulation to maximize conversion efficiency.

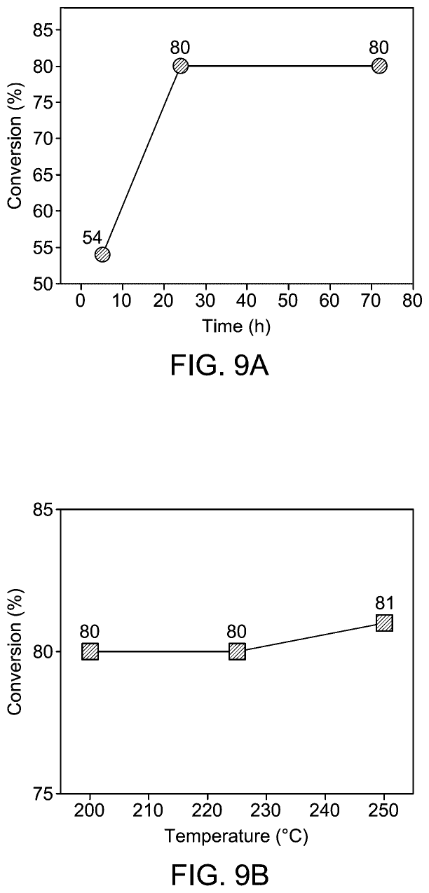

FIGS. 9A-9B depict insights into the dehydrogenation process of methylcyclohexane (MCH), illustrating the effects of reaction time and temperature on conversion efficiency. FIG. 9A represents the conversion percentage of MCH over time at a constant temperature. At the initial time point of 8 hours, the conversion rate is approximately 54%. This value increases overtime, reaching 80% at 24 hours. The conversion rate stabilizes at this level at 24 hours with no further increase in conversion at 72 h. This result indicates that equilibrium is reached, or the catalyst's activity remains constant beyond 24 hours.

FIG. 9B depicts the influence of temperature on the conversion percentage of MCH, with the data collected after a set reaction time. At 200 °C, the conversion rate is at 80%, indicating efficient dehydrogenation at this temperature. As the temperature increases to 230 °C, the conversion remains constant at 80%, demonstrating a robust catalytic performance across this temperature range; however, a slight increase in conversion to 81% is observed at 250 °C, indicating a slight improvement in the dehydrogenation efficiency at elevated temperatures. Together, FIGS. 9A-9B indicate that while reaction time plays a role in achieving high conversion rates, the dehydrogenation process is relatively insensitive to temperature variations within a temperature range of 200 to 250 °C. The slight increase in conversion at higher temperatures may be due to enhanced reaction kinetics, but the overall impact is minimal.

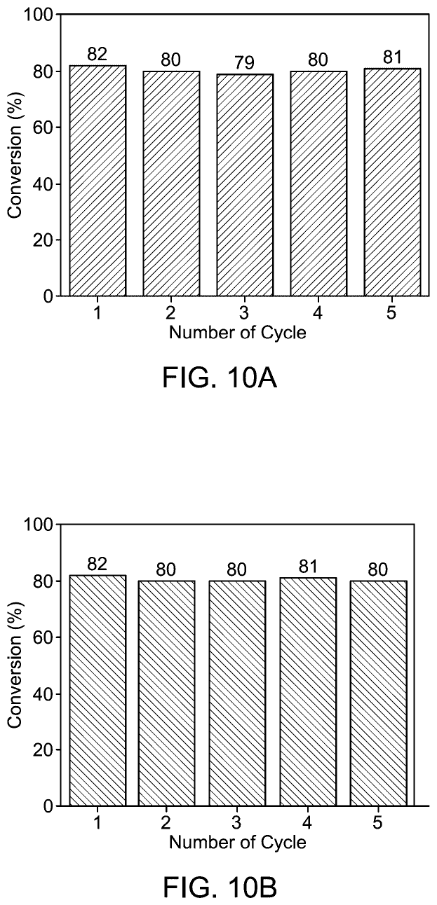

The reusability of the 0.5Rh-10% Mn/CeO₂ catalyst for the hydrogenation of toluene was evaluated over five consecutive cycles, as depicted in FIG. 10A. The conversion efficiency remained stable across all cycles, indicating good catalyst stability and reusability. Specifically, the conversion percentages were 82% for the first cycle, 80% for the second cycle, 79% for the third cycle, 80% for the fourth cycle, and 81% for the fifth cycle. The slight variations in conversion percentages, ranging from 79% to 82%, demonstrate a consistent catalytic performance. The average conversion efficiency over the five cycles was calculated to be 80.4%, underscoring the catalyst's robustness and minimal loss in activity across multiple uses.

Moreover, the reusability of the 0.5Rh-10% Mn/CeO₂ catalyst was evaluated through the dehydrogenation of methylcyclohexane (MCH) across five consecutive cycles, as shown in the FIG. 10B. The conversion efficiencies for each cycle were observed to be 82% in the first cycle, 80% in the second and third cycles, 81% in the fourth cycle, and 80% in the fifth cycle. These results indicate that the catalyst consistently maintains high activity, with conversion percentages ranging narrowly between 80% and 82%. The average conversion efficiency over these five cycles was calculated to be 80.6%, demonstrating the catalyst's stability and minimal loss of activity over repeated use.

These results elucidated that slight variations in conversion efficiencies across cycles for both hydrogenation and dehydrogenation experiments. The 0.5Rh-10% Mn/CeO₂ catalyst is robust and capable of sustaining performance in the hydrogenation of toluene and dehydrogenation of MCH. This consistent catalytic activity highlights the potential of the catalyst for practical applications in hydrogenation and dehydrogenation processes, where the durability and reusability of the catalyst are factors.

In the present disclosure, hydrogenation characteristics of toluene and dehydrogenation of MCH using various catalysts supported on CeO₂ was explored. Among the tested catalysts, 0.5% Rh10% Mn/CeO₂ demonstrated a 84% conversion of toluene with over 99% selectivity for MCH. The catalytic activity for the catalysts is as follows: 0.5Rh-10% Mn/CeO₂>0.5% Rh/CeO₂>0.5Rh-10% Co/CeO₂>0.5Rh-10% Ni/CeO₂>0.5Rh-10% Fe/CeO₂. Reaction conditions for hydrogenation of toluene were identified as 80 °C and 40 bars of pressure with a catalyst dosage of 10 mg. For the dehydrogenation process, 0.5% Rh10% Mn/CeO₂ facilitated 80% and 81% conversion of MCH at 200 °C and 250 °C, respectively. The catalyst also exhibited stability across five cyclic experiments for both hydrogenation and dehydrogenation, demonstrating potential for long-term use. These results underscore the effectiveness of 0.5% Rh10% Mn/CeO₂ in the reversible hydrogenation and dehydrogenation of toluene, highlighting its application in sustainable energy storage systems.

Numerous modifications and variations of the present disclosure are possible in light of the above teachings. It is therefore to be understood that within the scope of the appended claims, the disclosure may be practiced otherwise than as specifically described herein.

1: A method of hydrogenation, comprising:
contacting toluene with a catalyst and hydrogen gas in a reactor,
wherein the catalyst comprises a bimetallic cerium(IV) oxide (CeO₂) supported material,
wherein a first metal of the bimetallic CeO₂ supported material is rhodium in an amount of 0.01 to 0.05 percent by weight (wt. %),
wherein a second metal of the bimetallic CeO₂ supported material is selected from the group consisting of cobalt, iron, manganese, and nickel in an amount of 0.05 to 0.5 wt. %, and
hydrogenating the toluene to form a hydrogenated product.

2: The method of claim 1, wherein the contacting occurs at a temperature of 60 to 140 °C.

3: The method of claim 1, wherein the contacting occurs at a pressure of 20 to 60 bar.

4: The method of claim 1, wherein a weight ratio of the toluene to the catalyst is 15:1 to 20:1.

5: The method of claim 1, wherein 60 to 85% of the toluene is hydrogenated.

6: The method of claim 1, wherein the reactor comprises a stirrer.

7: The method of claim 6, wherein the stirrer has a rotational speed of up to 2500 rpm.

8: The method of claim 1, wherein at least 98% of the hydrogenated product is methylcyclohexane.

9: The method of claim 1, further comprising:
heating the hydrogenated product at a temperature of 180 to 270 °C to dehydrogenate the hydrogenated product.

10: The method of claim 9, wherein the heating occurs for at least 8 hours.

11: The method of claim 1, wherein the catalyst is in the shape of agglomerated particles having a particle circumference of 230 to 260 μm.

12: The method of claim 1, wherein the rhodium, the cobalt, the iron, the manganese, and the nickel have an oxidation state of zero.

13: The method of claim 1, wherein the second metal of the bimetallic CeO₂ supported material is manganese, and 80 to 90% of the toluene is hydrogenated.

14: The method of claim 1, wherein the second metal of the bimetallic CeO₂ supported material is cobalt, and 75 to 80% of the toluene is hydrogenated.

15: The method of claim 1, wherein the second metal of the bimetallic CeO₂ supported material is nickel, and 65 to 70% of the toluene is hydrogenated.

16: The method of claim 1, wherein the second metal of the bimetallic CeO₂ supported material is iron, and 62 to 66% of the toluene is hydrogenated.

17: The method of claim 9, further comprising:
repeating the contacting to form the hydrogenated product;
repeating the heating to dehydrogenate the hydrogenated product,
wherein the repeating occurs at least three times,
wherein forming the hydrogenated product has a conversion rate of at least 80%,
wherein dehydrogenating the hydrogenated product has a conversion rate of at least 80%,
wherein conversion rate is based on weight of the toluene and the hydrogenated product.

18: The method of claim 11, wherein the agglomerated particles comprise nanoparticles having a longest dimension of 10 to 100 nm.

19: The method of claim 9, wherein the heating occurs for at least 24 hours and at least 80% of the hydrogenated product is dehydrogenated.

20: The method of claim 1, wherein the catalyst is made by a process comprising:
dissolving a rhodium salt and a salt selected from the group consisting of a cobalt salt, an iron salt, a manganese salt, and a nickel salt in water to form a first solution;
adding cerium oxide to the first solution to form a second solution;
heating the second solution to a temperature of 50 to 70 °C for 60 to 120 minutes; and
adding a base to the second solution to form the catalyst.
